(19) United States
(12) Patent Application Publication
(10) Pub. No.: US 2017/0072182 A1
(43) Pub. Date: Mar. 16, 2017

(54) NEEDLELESS CONNECTOR WITH FLEXIBLE VALVE

(71) Applicant: LILY MEDICAL CORPORATION, Miaoli County (TW)

(72) Inventors: HSIEN-CHIH TSAI, TAIPEI CITY (TW); YUNG-HUNG CHIH, NEW TAIPEI CITY (TW); CHIH-WEN LI, TAOYUAN CITY (TW)

(21) Appl. No.: 14/960,494

(22) Filed: Dec. 7, 2015

(30) Foreign Application Priority Data
Sep. 16, 2015 (TW) 104214976

(51) Int. Cl. A61M 39/12; A61M 39/24; A61M 5/31

(52) U.S. Cl. CPC A61M 39/12; A61M 5/31; A61M 39/24; A61M 2005/3128; A61M 2039/2433

(57) ABSTRACT

A needleless connector comprises a lid, a containing seat, and a flexible valve. One end of the lid is connected to one end of the containing seat, the interior of the lid and the interior of the containing seat form a space. The flexible valve is disposed in the space, and the flexible valve has a closed end and an opening end. The closed end is disposed in the inserting hole of the lid, and the opening end toward the closed end is formed inward with a groove. The flexible valve is defined in sequence as a head portion, a shoulder portion, a bending portion, and a supporting portion from the closed end to the opening end. The outer diameter of the head portion is smaller than the outer diameter of the bending portion. A shoulder portion is connected between the head portion and the bending portion.

### BACKGROUND OF THE INVENTION

1. Field of the Invention

The present invention is related to a medical syringe connector structure, in particular, to a needleless connector with flexible valve for injecting potion by syringe without steel needle.

2. Description of Related Art

Medical patients are injected with all kinds of medicinal fluids as needed, and one of the most common ways is to be injected is with IV drip. The doctor adds other medicinal fluids for patients through the feeder connected to a tube of the IV drip, so the medicinal fluid is injected through the tube and then into the IV drip.

The conventional feeder includes a lid, a flexible valve and a containing seat. In general, the flexible valve is utilized to seal the injecting opening of the feeder. When a syringe is inserted into the injecting opening of the feeder, the flexible valve is abutted and bent into the lid and the containing seat, so the medicinal fluid of the syringe is injected into a space formed by the interior of the lid and the interior of the containing seat. The medicinal fluids are guided by the containing seat into the tube of IV drip. When the injection is completed, the syringe is retreated from the lid and the flexible valve goes back to normal by the elastic force. However, the flexible valve often gets stuck in the lid and is not restored to normal, so the medicinal fluid cannot be injected into accordingly when the medical staff operates the feeder.

In summary, the inventor of this instant disclosure has contributed to research and developed the needleless connector of the instant disclosure to overcome the abovementioned drawbacks.

### SUMMARY OF THE INVENTION

The object of the instant disclosure is to provide a needleless connector with flexible valve to resolve the jamming or reposition issues of the traditional flexible valve of the feeder.

The needleless connector according to the present invention includes a lid, a containing seat, and a flexible valve. The lid has an inserting hole and an opening. The containing seat has a connecting opening and an outlet, the connecting opening engages with the outlet, the interior of the lid and the interior of the containing seat form a space. The flexible valve is disposed inside the space and has a closed end and an opening end. The opening end toward the closed end is formed inward with a groove, and the side wall of the groove does not have any hole. The axial cross-section of the flexible valve is formed symmetrically. The closed end is formed with a head portion, the head portion toward the opening end is formed with a shoulder portion, the shoulder portion toward the opening end is formed with a bending portion. The shoulder portion is connected between the head portion and the bending portion. The bending portion toward the opening end is formed with a supporting portion. The outer diameter of the head portion is smaller than the outer diameter of the bending portion, the maximum thickness section of the shoulder portion is defined as a first wall thickness, and the minimum thickness section of the bending portion is defined as a second wall thickness, wherein the second wall thickness is not more than 0.7 times that of the first wall thickness.

The present invention further provides a flexible valve for a needleless connector. The flexible valve has a closed end and an opening end, the opening end toward the closed end is formed inward with a groove, and the side wall formed with the groove does not have any hole. The axial cross-section of the flexible valve is formed symmetrically. The closed end is formed with a head portion, the head portion toward the opening end is formed with a shoulder portion, the shoulder portion toward the opening end is formed with a bending portion, the bending portion toward the opening end is formed with a support portion. The outer diameter of the head portion is smaller than the outer diameter of the bending portion The maximum thickness section of the shoulder portion is defined as a first wall thickness, and the minimum thickness section of the bending portion is defined as a second wall thickness, wherein the second wall thickness is not more than 0.7 times that of the first wall thickness.

The present invention has the advantages that the thickness of the flexible valve is varied. When the external syringe is abutted against the flexible valve, the lid and the containing seat are bent, so the potion is guided by the containing seat and flow through the outlet (are injected into the pipe of the IV drip), and the flexible valve is restored and sealed the inserting hole while the syringe is pulled away from the inserting hole of the lid.

For further understanding of the instant disclosure, reference is made to the following detailed description illustrating the embodiments and examples of the instant disclosure. The description is for illustrative purpose only and is not intended to limit the scope of the claim.

### BRIEF DESCRIPTION OF THE DRAWINGS

FIGS. 1 and 2 illustrate an exploded view of the first embodiment of the instant disclosure.

FIGS. 3 and 4 illustrate a cross-sectional view of the flexible valve of the first embodiment of the instant disclosure.

FIGS. 5 and 6 illustrate a cross-sectional view of a containing seat of a first embodiment of the instant disclosure.

FIG. 7 illustrates a perspective view of a first embodiment of the instant disclosure.

FIGS. 8 and 9 illustrate a cross-sectional view of a first embodiment of the instant disclosure.

FIGS. 10 and 11 illustrate an example of a needleless connector in use, in accordance with a first embodiment of the instant disclosure.

FIG. 12 illustrates a cross-sectional view of a flexible valve of the second embodiment of the instant disclosure.

FIGS. 13 and 14 illustrate a cross-sectional view of a containing seat of the third embodiment of the instant disclosure.

FIG. 15 illustrates a cross-sectional view of a containing seat of the fourth embodiment of the instant disclosure.

FIG. 16 illustrates a cross-sectional view of the fifth embodiment of the instant disclosure.

FIG. 17 illustrates an exploded view of the sixth embodiment of the instant disclosure.

FIG. 18 illustrates a perspective view of the sixth embodiment of the instant disclosure.

### DETAILED DESCRIPTION OF THE PREFERRED EMBODIMENTS

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

### First Embodiment

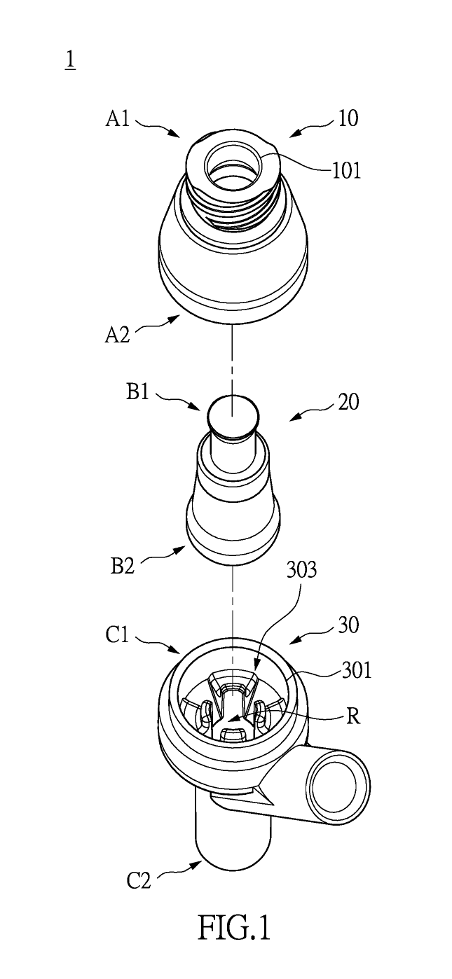

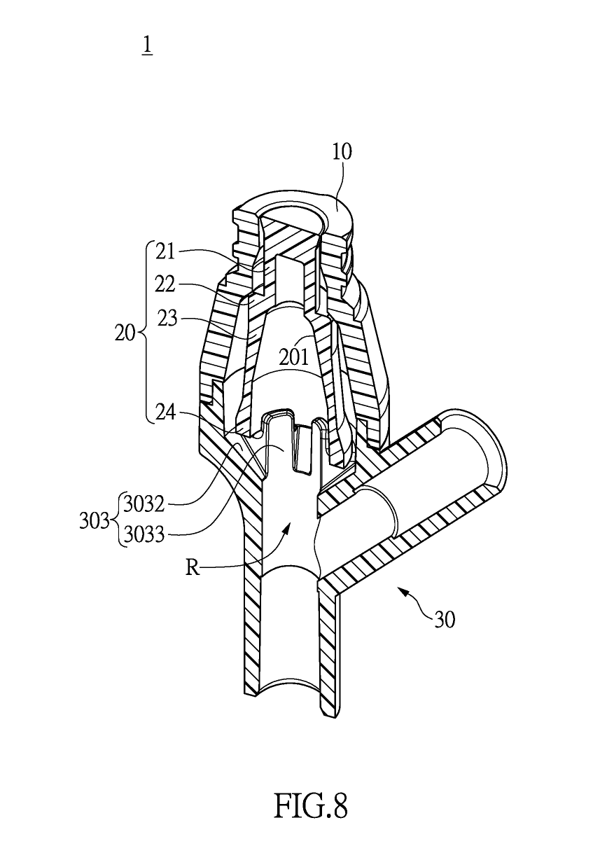

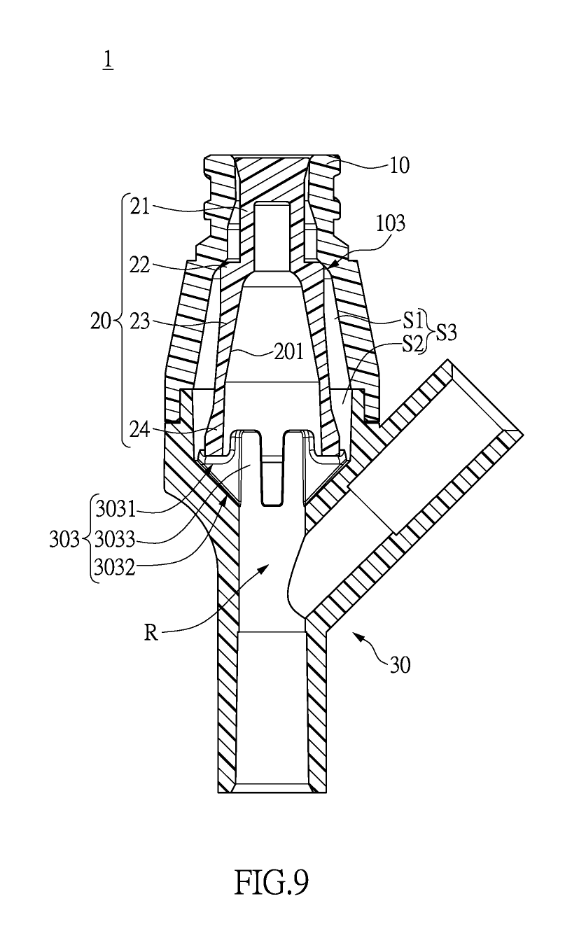

The first embodiment according to the present invention is illustrated in FIGS. 1 to 11. With reference FIGS. 1, 2, 8, and 9, the present invention provides a needleless connector including a lid 10, a flexible valve 20 and a containing seat 30. One end of the lid 10 is connected to one end of the containing seat 30. The interior of the lid 10 and the interior of the containing seat 30 form a space S3, and the flexible valve 20 is installed in the space S3. The flexible valve 20 is actually spliced onto the containing seat 30, and the lid 10 is jointed and fixed with the containing seat 30 by splicing.

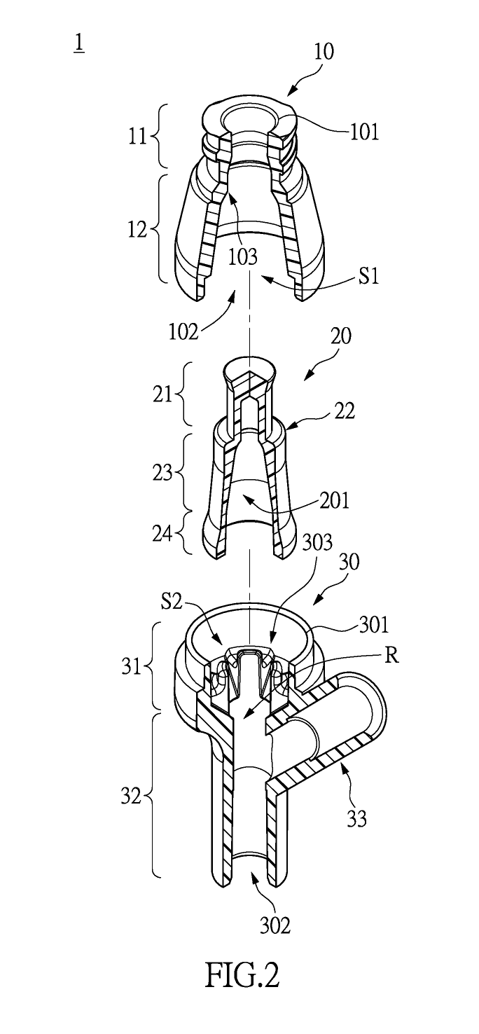

With reference to FIG. 1 and FIG. 2, the lid 10 is hollow and two ends of the lid 10 are defined as an inserting end A1 and joining end A2 respectively. The interior of the lid 10 forms a space S1. The inserting end A1 of the lid 10 is formed with an inserting hole 101, the joining end A2 of the lid 10 is formed with an opening 102. The inserting hole 101 and the opening 102 penetrate through the lid 10 and are connected to the space S1. The interior of the lid 10 is formed with a projection 103 adjacent to the inserting end A1. It is worth mentioning that the shape of the lid 10 is divided into a guiding portion 11 and expanding portion 12. The exterior and the interior of the guiding portion 11 is essentially straight. The guiding portion 11 is utilized to provide a path for guiding an external syringe 2 that inserts through the inserting hole 101 of the lid 10. The exterior and the interior of the expanding portion 12 is essentially with a cone shape (the farther apart from inserting end A1, the bigger the diameter). The interior of the expanding portion 12 is utilized to provide an activity space for the bending of the flexible valve 12. The abovementioned projection 103 is formed between the guiding portion 11 and expanding portion 12.

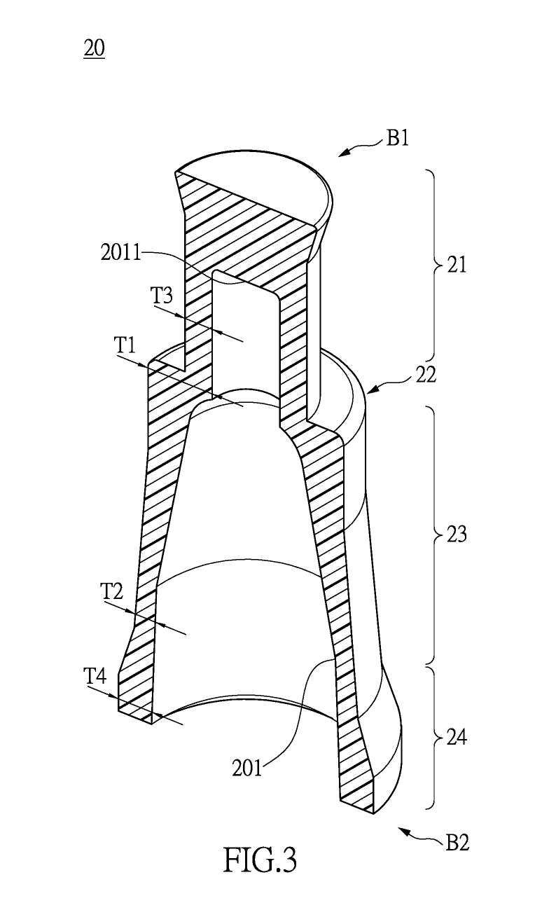

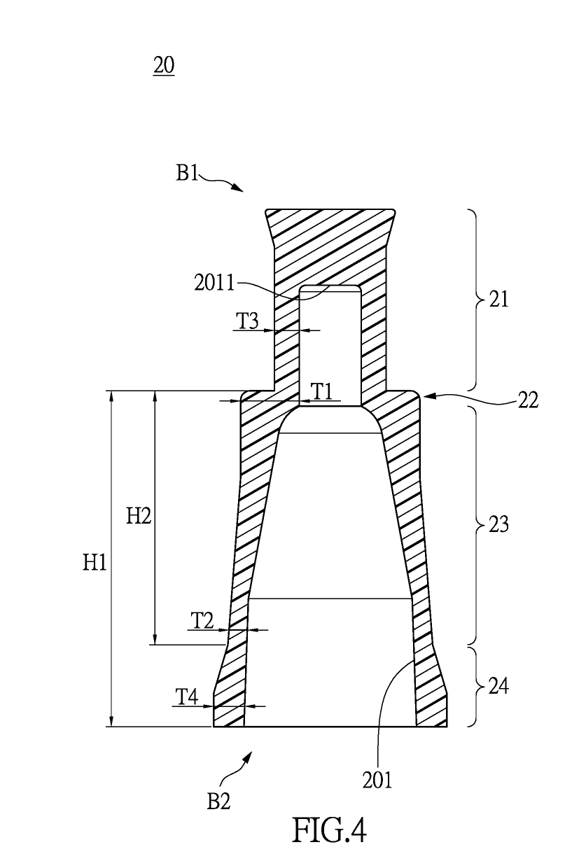

With reference to FIG. 3 and FIG. 4, the flexible valve 20 has a closed end B1 and an opening end B2. The opening end B2 is formed inward toward the closed end B1 with a groove 201. The groove 201 is only connected to the outside though the opening end B2. In other words, the side wall of the flexible valve 20 formed by the groove 201 does not have any penetrated hole. The flexible valve 20 has a head portion 21, a shoulder portion 22, a bending portion 23, and a supporting portion 24 in series from the closed end B1 to the opening end B2. In other words, the closed end B1 is formed with a head portion 21, the opening end B2 is formed with a supporting portion 24. The head portion 21 toward the opening end B2 is formed with a shoulder portion 22, and the shoulder portion 22 toward the opening end B2 is formed with a bending portion 23. The bending portion 23 is connected the supporting portion 24. The outer diameter of the head portion 21 is smaller than the outer diameter of the bending portion 23. A shoulder portion 22 is formed between the head portion 21 and bending portion 23. The head portion 21 and the bending portion 23 is essentially with a tube shape. With reference to FIG. 4, in this embodiment, the axial cross-section of the flexible valve 20 is symmetrical.

The maximum thickness section of the shoulder portion 22 is defined as a first wall thickness T1. The minimum thickness section of the bending portion 23 is defined as a second wall thickness T2. The second wall thickness T2 is not more than 0.7 times that of the first wall thickness T1. Preferably, the second wall thickness T2 is not less than 0.3 times that of the first wall thickness T1. The relationship of the first wall thickness T1 and the second wall thickness T2 is represented in 0.3 T1≦T2≦0.7 T1. For instance, suppose the first wall thickness T1 is 1.5 mm, and the second wall thickness T2 is from 0.45 mm to 1.05 mm. The maximum thickness section of the shoulder portion 22 is between the shoulder portion 22 and the head portion 21, and the thickness of the shoulder portion 22 is gradually reduced. In other words, the thickness of the shoulder portion 22 is gradually reduced from the closed end B1 of the flexible valve 20 toward the opening end B2 of the flexible valve 20.

The groove 201 of the flexible valve 20 has a bottom 2011 corresponding to the head portion 21. The section adjacent to the closed end B1 of the head portion 21 is solid, and the rest of the head portion 21 is hollow. The hollow section of the head portion 21 has a thickness defined as a third wall thickness T3. Preferably, the third wall thickness T3 is not more than 0.7 time of the first wall thickness T1. The third wall thickness T3 is not less than 0.1 time of the first wall thickness T1. In other words, the preferred range of the third wall thickness T3 is from 0.1 times the first wall thickness T1 to 0.7 times that of the first wall thickness T1. The relationship of the third wall thickness T3 and the first wall thickness T1 is represented in 0.1 T1≦T2≦0.7 T1. For instance, suppose the first wall thickness T1 is 1.5 mm, and the third wall thickness T3 is from 0.15 mm to 1.05 mm.

A distance from the shoulder portion 22 to the opening end 22 is defined as a first height H1. A distance from the shoulder portion 22 to a minimum section of the bending portion 23 is defined as a second height H2. Preferably, the second height H2 is not more than three fifths of the first height H1, and the second height H2 is not less than one fifth. Namely, the relationship of the first height H1 and the second height H2 is represented in ⅕ H1≦H2≦⅗ H1. For instance, suppose the first height H1 is 11 mm, and the second height H2 is from 2.2 mm to 6.6 mm. In practice, the thickness of the bending portion 23 is gradually increased toward both ends.

Preferably, the thickness of the supporting portion 24 of the flexible valve 20 is from 0.4 times that of the first wall thickness T1 to 1.4 times the first wall thickness T1. The supporting portion 24 of the flexible valve 20 provides good strength so the flexible valve 20 is installed firmly in conduction structure 303 (as shown in FIG. 2). The flexible valve 20 is bent at the bending portion 23 while the external syringe is abutted against the flexible valve 20.

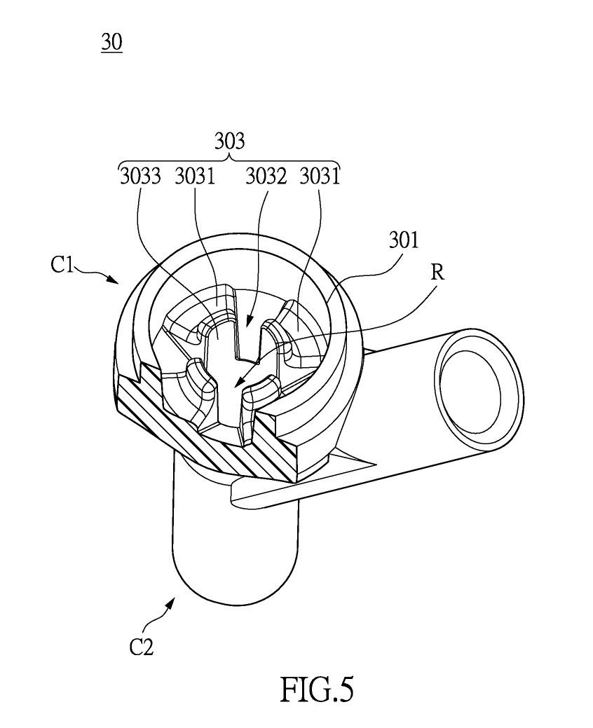

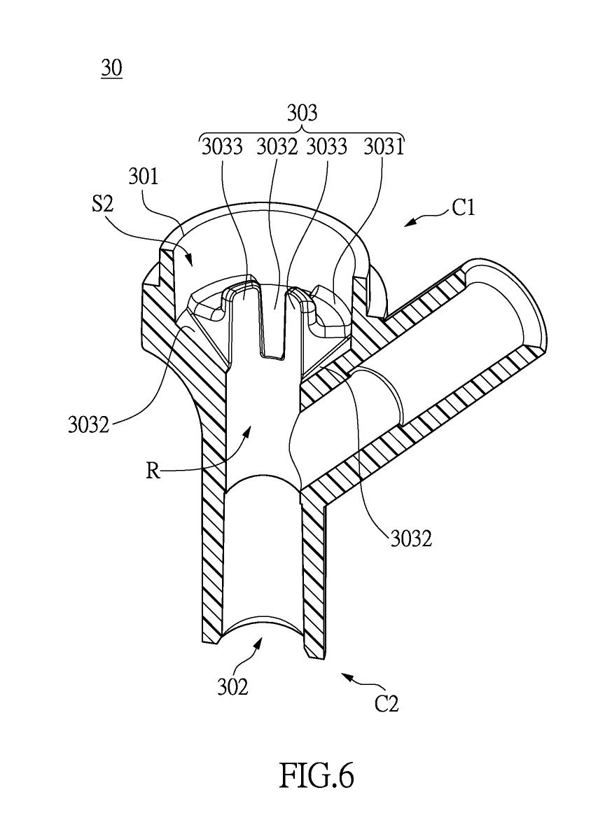

With reference to FIG. 1, FIG. 5, and FIG. 6, the containing seat 30 is hollow, and two ends of the containing seat 30 are defined as a connecting end C1 and a deriving end C2 respectively. The interior of the containing seat 30 forms a space S2. The connecting end C1 of the containing seat 30 is formed with a connecting opening 301, the deriving end C2 is formed with an outlet 302. The connecting opening 301 and the outlet 302 penetrate the contain seat 30. Interior of the containing seat 30 adjacent to the connecting end C1 is formed with a conduction structure 303. The center of the conduction structure 303 is formed with a central tunnel R. The central tunnel R connects the space S2 and outlet 302. The conduction structure 303 and the central tunnel R are utilized to guide the medicinal fluid in the needleless connector 1, and the medicinal fluid flows from the outlet 302. In this embodiment, the containing seat 30 is roughly divided into a seat portion 31, guiding portion 32 and Y-branch portion 33 based on the shape of the containing seat 30. The outer and inner diameter of the seat portion 31 are roughly corresponding to the outer and inner diameter of the joining end A2 of the lid 10, so the seat portion 31 of the containing seat 30 engages with the joining end A2 of the lid 10, and the conduction structure 303 is formed inside the seat portion 31. The guiding portion 32 is with a tube shape, and connected to the pipe of the corresponding IV drip. The guiding portion 32 is extended gradient outward to form the Y-branch portion 33, and the Y-branch portion 33 is a tunnel and configured to be injected with another medicinal fluid.

More specifically, the conduction structure 303 has four bearing portions 3031, four guiding tunnels 3032, and four positioning portions 3033. Interior of the containing seat 30 toward the center extends to form the bearing portions 3031, and the guiding tunnels 3032 are formed between two bearing portions 3031. One end of the bearing portion 3031 away from the inner sidewall of the containing seat 30 is formed with the positioning portion 3033 toward the joining end A2. The positioning portion 3033 is approximately at the center of the conduction structure 303, and forms the central tunnel R. Preferably, the guiding tunnel 3032 is a tilt structure and extended from the inner sidewall of the containing seat 30 toward the deriving end C2. The number of the bearing portion 3031, the guiding tunnel 3032, and the positioning portion 3033 are adjustable as required, the number is not limited to 4.

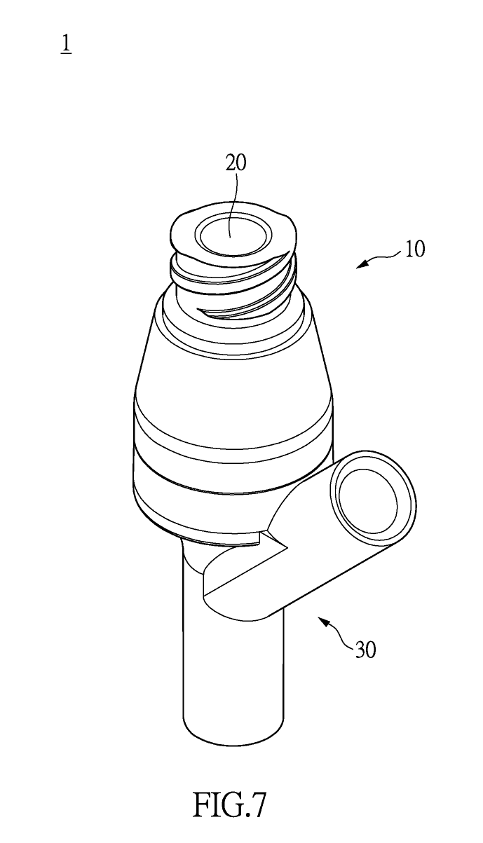

With reference to FIG. 7 to FIG. 9, the lid 10 engages with the containing seat 30, and the space S1 of the lid 10 is connected to the space S2 of the containing seat 30 to form a space S3, and the flexible valve is disposed in the space S3 accordingly. More specifically, part of the head portion 21 is disposed in the inserting hole 101 of the lid 10, so the head portion 21 is sealed in the inserting hole 101. A gap is formed between the outside of the head portion 21 and the inner sidewall of the guiding portion 11 (shown in FIG. 2), configured to provide an activity space for the bending of the head portion 21 when abutted against by an external syringe. The outside of the shoulder portion 22 is abutted against the projection 103 of the lid 10 and seals the space between the head portion 21 and inner sidewall of the lid 10. A gap is formed between the bending portion 23 of the flexible valve 20, outside of the supporting portion 24, and the inner sidewall of the lid 10. Accordingly, the needleless connector 1 is made tighter by the inserting of the flexible valve 20 and the abutting between the shoulder portion 22 and projection 103. The opening end B2 (as shown in FIG. 1) of the flexible valve 20 is nested in the positioning portion 3033 of the containing seat 30. The opening end B2 of the flexible valve 20 is disposed on the bearing portion 3031 of the conduction structure 303. The guiding tunnel 3032 of the conduction structure 303 is disposed below the flexible valve 20 and is not contacted with the flexible valve 20.

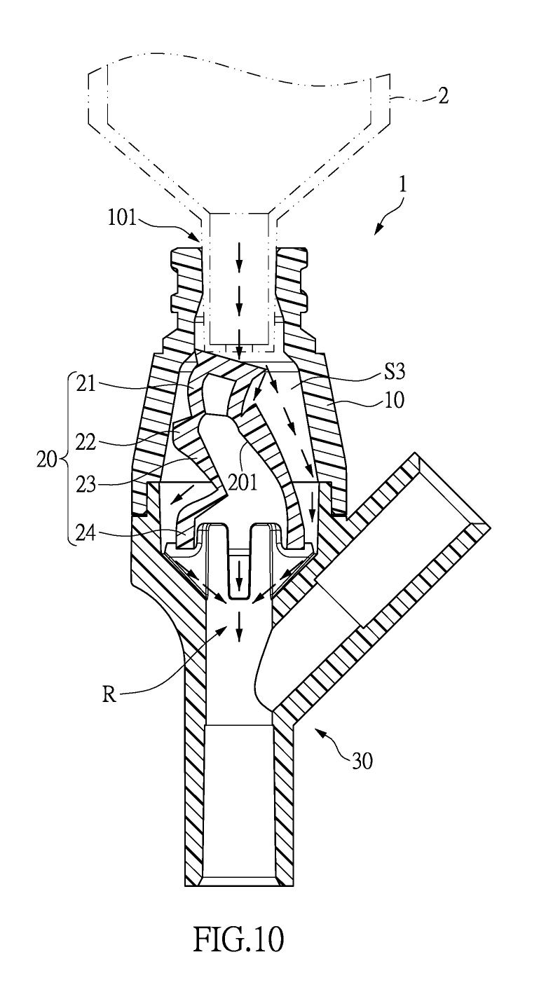

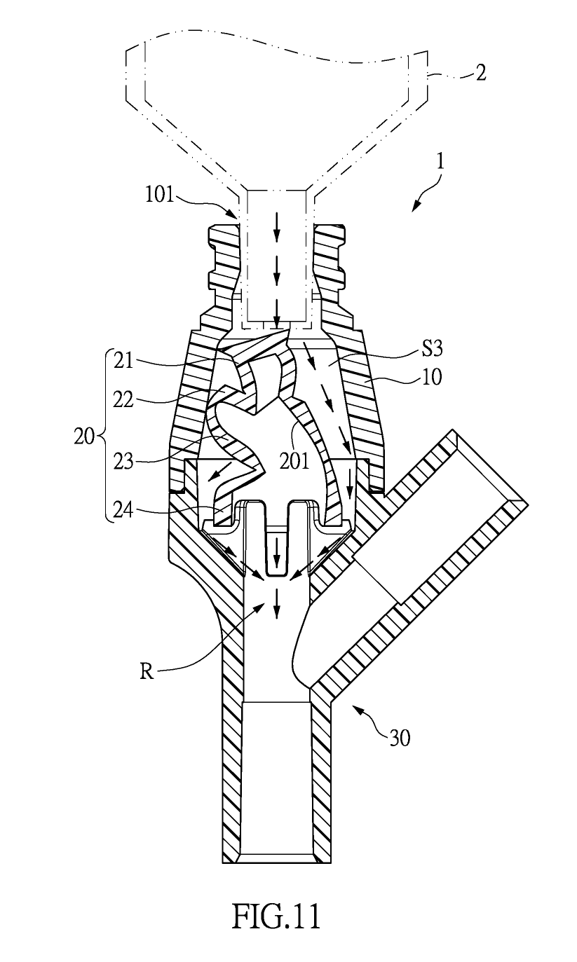

With reference FIG. 10 and FIG. 11, the syringe 2 is inserted from the inserting hole 101 of the lid 10, the flexible valve 20 is abutted against by the syringe 2 and begins to buckle, and is disposed into the space S3 formed by the interior of the lid 10 and the interior of the containing seat 30. More specifically, the syringe 2 is abutted against the flexible valve 20, and the head portion 21 of the flexible valve 20 is moved and bent toward the containing seat 30, so the shoulder portion 22 is not abutted against the projection 103 of the lid 10 (namely, the shoulder portion 22 is retreated from the projection 103), and the head portion 21 no longer seals the space between the inserting hole 101 and the projection 103. The outer sidewall of the flexible valve 20, the lid 10, and the inner sidewall of the containing seat 30 form a flow path connected to the outlet 302, so the medicinal fluid of the syringe 2 goes through the flow path (space S3), the guiding tunnel 3032 of the conduction structure 303, and the central tunnel R, and then is discharged from the outlet 302 of the containing seat 30.

It is worth to mention that the hollow part of the head portion 21 has the same thickness, so the syringe 2 is abutted against the head portion 21, and the head portion 21 could be bent toward the direction as shown in FIG. 10 (the left side of the diagram) or shown in FIG. 11 (the right side of the diagram). The minimum thickness section of the bending portion 23 of the flexible valve 20 could be bent to the right (as shown in FIG. 10) or to the left (as shown in FIG. 11).

With reference FIG. 6 and FIG. 10, each guiding tunnel 3032 is formed between two bearing portions 3031, and each guiding tunnel 3032 is a sloping downward structure. The supporting portion 24 of the flexible valve 20 is disposed on the bearing portion 3031. The groove 201 of the flexible valve 20 is nested into the positioning portion 3033. A gap is formed between the bending portion 23 and the inner sidewall of the lid 10 when the flexible valve 20 is not abutted against by the syringe 2. In practice, when the flexible valve 20 is abutted against by the syringe 2, it does not matter which direction the flexible valve 20 is bent toward (even if the flexible valve 20 is stuck in the lid 10), the portion of the syringe 2 could move smoothly between the flexible valve 20 and inner sidewall of the lid 10 and through the guiding tunnel 3032, and toward the deriving end C2.

### Second Embodiment

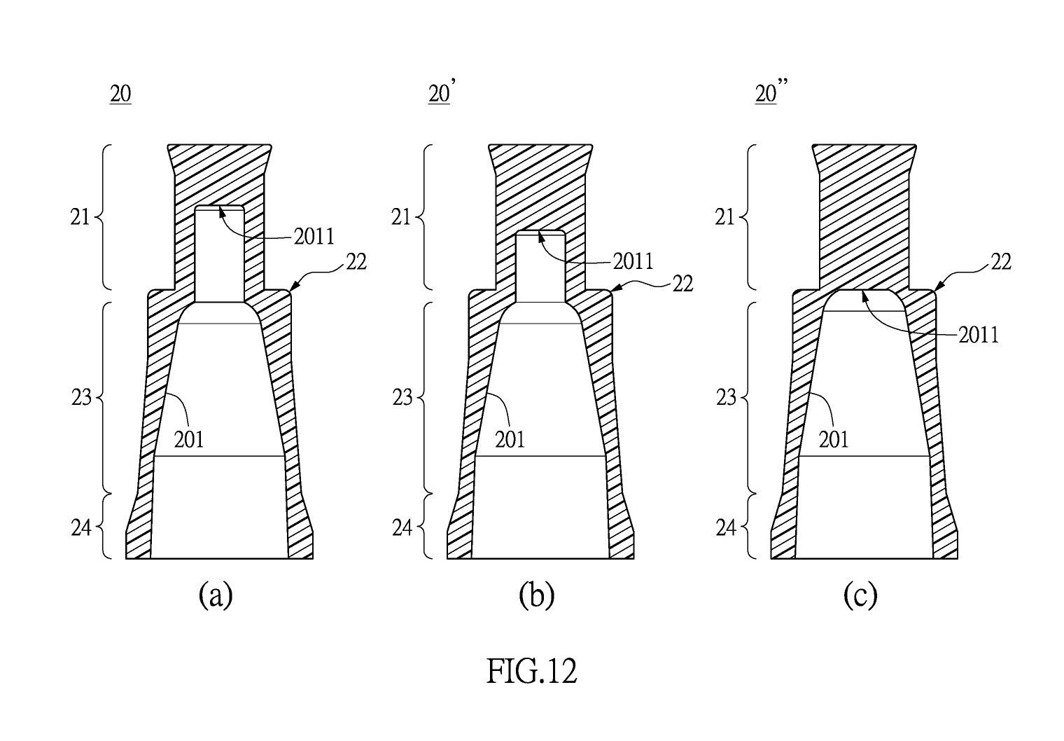

FIG. 12 illustrates the second embodiment of the present invention. FIG. 12(a) illustrates the flexible valve 20 of the first embodiment, the bottom 2011 of the groove 201 of the flexible valve 20 is corresponding to the head portion 21. With reference to FIG. 12(b), the position of the bottom 2011 of the groove 201 is corresponding to half of the axial length of the head portion 21. With reference to FIG. 12(c), the position of the bottom 2011 of the groove 201 is corresponding to the shoulder portion 22, in other words, the head portion 21 is solid.

### Third and Fourth Embodiment

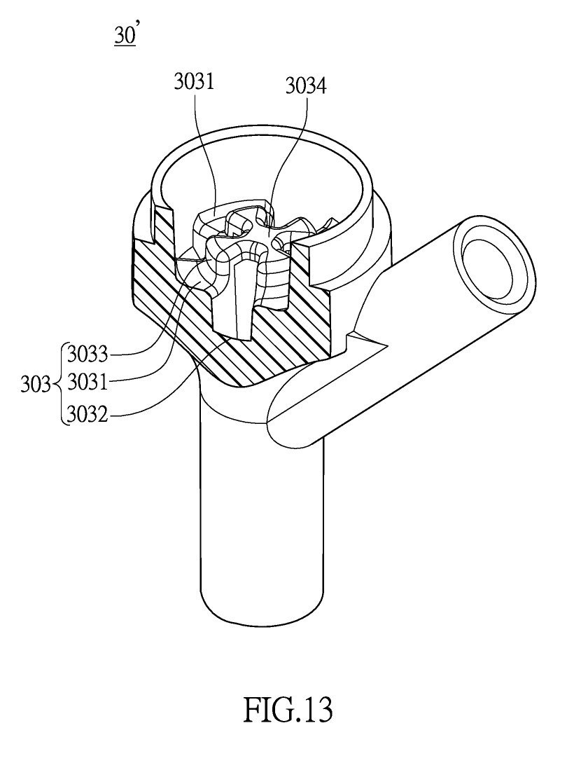

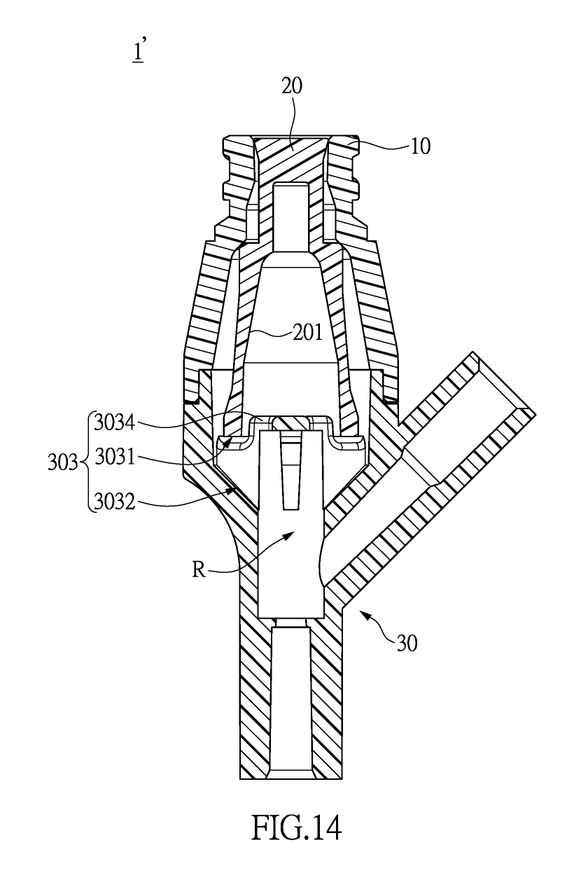

FIG. 13 and FIG. 14 illustrate the third embodiment of the present invention. As shown in the figures, the containing seat 30′ of the needleless connector 1′ includes a plurality of positioning portions 3033, and one end (away from the bearing portion 3031) of the positioning portions 3033 are jointed together and form a platform 3034, and the platform 3034 covers part of the central tunnel R. While the syringe 2 (as shown in FIG. 10) is retreated from the lid 10 (as shown in FIG. 10), the rest of the fluid of the containing seat 30 is not recirculated easily back to the groove 201 of the flexible valve 20. Moreover, the guiding tunnels 3032 are sloped toward the central tunnel R and configured to avoid the rest of the medicinal fluid being retrograded back into the space S3.

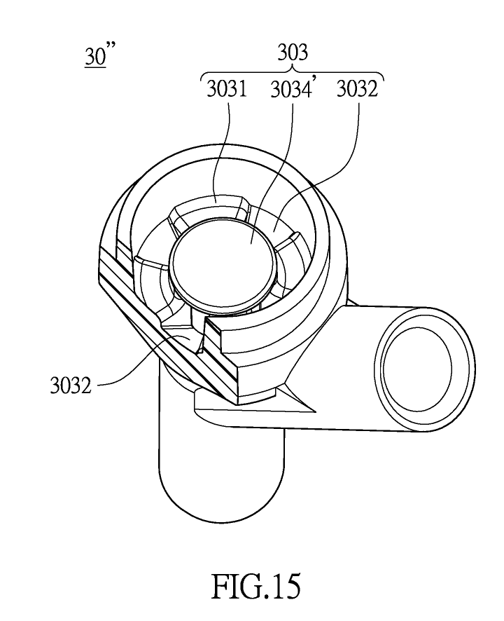

With reference to FIG. 15, the platform 3034′ of the conduction structure 303 of the containing seat 30″ is a dish-like structure, so the conduction structure 303 is configured to avoid the rest of medicinal fluid being retrograded back.

### Fifth Embodiment

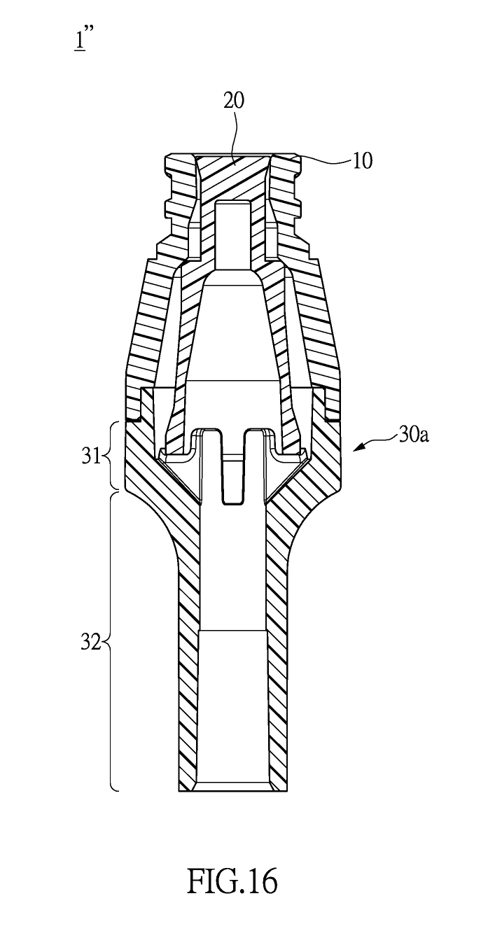

Please refer to FIG. 16. In the fifth embodiment of the invention, a containing seat will be explained in the following as the rest of other elements are the same as the aforementioned embodiment. A containing seat 30a of the needleless connector 1″ does not include the Y-branch portion as shown in FIG. 2, and the containing seat 30a of the needleless connector 1″ simply has a seat portion 31 and guiding portion 32.

### Sixth Embodiment

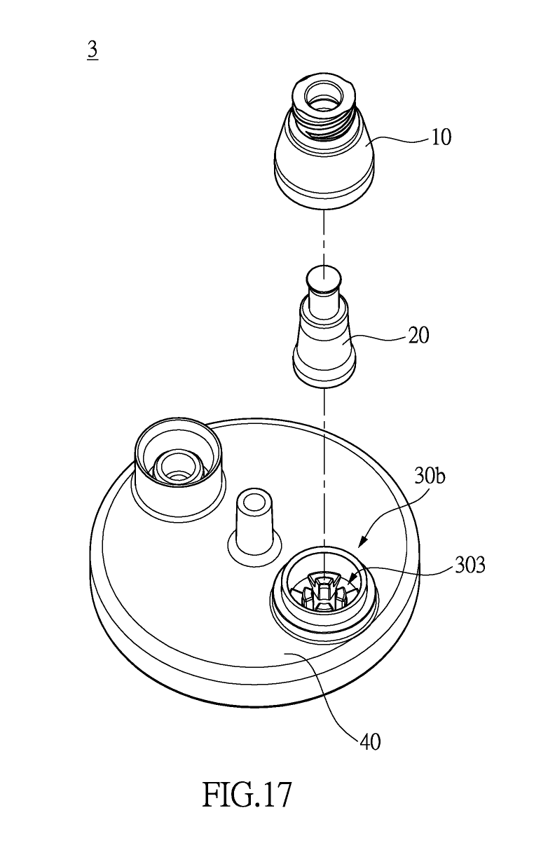

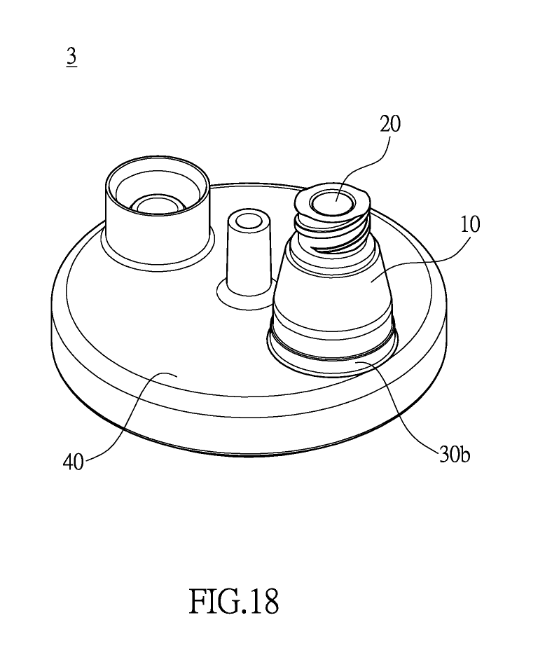

Please refer to FIG. 17 and FIG. 18. In the sixth embodiment, a containing seat 30b of the needleless connector 3 is different from the containing seat in the aforementioned embodiment. The containing seat 30b in this embodiment does not include a guiding portion 32 and Y-branch portion, and the containing seat 30b is formed directly on a top cover 40. More specifically, the top cover 40 is fixed on the top of a regulator (not shown) of a IV drip, and the doctors or nurses inject the medicinal fluid into the regulator directly.

The descriptions illustrated supra set forth simply the preferred embodiments of the instant disclosure; however, the characteristics of the instant disclosure are by no means restricted thereto. All changes, alterations, or modifications conveniently considered by those skilled in the art are deemed to be encompassed within the scope of the instant disclosure delineated by the following claims.

What is claimed is:

1. A needleless connector, comprising:
a lid, having an inserting hole and an opening;
a containing seat, having a connecting opening and an outlet, said connecting opening engaged with the outlet, the interior of the lid and the interior of the containing seat forming a space; and
a flexible valve, disposed inside the space and having a closed end and an opening end, the opening end formed inward toward the closed end with a groove, and a side wall formed with the groove having not any penetrated hole, and the axial cross-section of the flexible valve formed symmetrically, the closed end formed with a head portion, the head portion toward the opening end formed with a shoulder portion, the shoulder portion toward the opening end formed with a bending portion, the bending portion toward the opening end formed with a supporting portion, the outer diameter of the head portion smaller than the outer diameter of the bending portion, the maximum thickness section of the shoulder portion defined as a first wall thickness, and the minimum thickness section of the bending portion defined as a second wall thickness, wherein the second wall thickness is not more than 0.7 times that of the first wall thickness.

2. The needleless connector according to claim 1, wherein at least one portion of the head portion is inserted and seals the inserting hole, an interior of the lid is formed with a projection adjacent to the inner sidewall of the inserting hole, the outer portion of the shoulder portion is abutted against the projection and seals the space between the head portion and the inner sidewall of the lid, and a gap is formed between the bending portion, outside of the supporting portion, and the inner sidewall of the lid, interior of the contain seat is formed with a conduction structure adjacent to the connecting opening, the central region of the conduction structure is formed with a central tunnel, the central tunnel, the space and the outlet are interconnecting, when a syringe is inserted through the inserting hole and abutted against the head portion, the shoulder portion of the flexible valve is retreated from the projection, and the outer sidewall of the flexible valve and the sidewall of the space are formed with a tunnel, and the medicinal fluid of the syringe is drained out of the outlet through the tunnel, the conduction structure, and the central tunnel.

3. The needleless connector according to claim 2, wherein a bottom of the groove is corresponding to the shoulder portion of the flexible valve, and the head portion is solid.

4. The needleless connector according to claim 2, wherein a bottom of the groove is corresponding to the shoulder portion of the flexible valve, and a section adjacent to the closed end of the head portion is solid, whereas the rest of the head portion is hollow, the hollow section of the head portion has a third wall thickness, and the third wall thickness is not more than 0.7 times that of the first wall thickness.

5. The needleless connector according to claim 4, wherein the third wall thickness is not less than 0.1 times that of the first wall thickness.

6. The needleless connector according to claim 5, wherein the second wall thickness is not less than 0.3 times that of the first wall thickness.

7. The needleless connector according to claim 6, wherein a distance from the shoulder portion of the flexible valve to the opening end is defined as a first height, a distance from the shoulder portion to a minimum section of the bending portion is defined as a second height, the second height is between from ⅕ of the first height to ⅗ of the first height.

8. The needleless connector according to claim 7, wherein the thickness of the supporting portion of the flexible valve is between from 0.4 times that of the first wall thickness to 1.4 times that of the first wall thickness.

9. The needleless connector according to claim 7, wherein the conduction structure has a plurality of positioning portions, guiding tunnels and bearing portions, interior of the conduction structure toward the center formed with the bearing portions, the guiding tunnels are formed between the two bearing portions, one end of each the bearing portions away from the inner sidewall of the containing seat is extended toward the joining end to form the positioning portion, wherein the opening end of the flexible valve is nested into the positioning portion, and disposed in the bearing portion of the conduction structure.

10. The needleless connector according to claim 9, wherein the inner sidewall of the containing seat is extended toward the deriving end to form the guiding tunnel, and the guiding tunnel is a slope structure.

11. The needleless connector according to claim 10, wherein each one end of the positioning portion away from the bearing portion is connected to each other to form a platform structure.

12. The needleless connector according to claim 1, wherein the thickness of both sides of the section with minimal thickness of the bending portion is increased respectively toward the opening end and the closed end.

13. A flexible valve of a needleless connector, having a closed end and an opening end, the opening end toward the closed end formed inward with a groove, and a side wall formed with the groove having not any penetrated hole, and the axial cross-section of the flexible valve formed symmetrically, the closed end formed with a head portion, the head portion toward the opening end formed with a shoulder portion, the shoulder portion toward the opening end formed with a bending portion, the bending portion toward the opening end formed with a support portion, the outer diameter of the head portion smaller than the outer diameter of the bending portion, the maximum thickness section of the shoulder portion defined as a first wall thickness, and the minimum thickness section of the bending portion defined as a second wall thickness, wherein the second wall thickness is not more than 0.7 times that of the first wall thickness.

14. The flexible valve of a needleless connector according to claim 13, wherein a bottom of the groove is corresponding to the shoulder portion of the flexible valve, and the head portion is solid.

15. The flexible valve of a needleless connector according to claim 13, wherein a bottom of the groove is corresponding to the shoulder portion of the flexible valve, and a section adjacent to the closed end of the head portion is solid, whereas the rest of the head portion is hollow, the hollow section of the head portion has a third wall thickness, and the third wall thickness is not more than 0.7 times that of the first wall thickness.

16. The flexible valve of a needleless connector according to claim 15, wherein the third wall thickness is not less than 0.1 times that of the first wall thickness.

17. The flexible valve of a needleless connector according to claim 15, wherein the second wall thickness is not less than 0.3 times that of the first wall thickness.

18. The flexible valve of a needleless connector according to claim 17, wherein a distance from the shoulder portion of the flexible valve to the opening end is defined as a first height, a distance from the shoulder portion to a minimum thickness section of the bending portion is defined as a second height, and the second height is between from ⅕ of the first height to ⅗ of the first height.

19. The flexible valve of a needleless connector according to claim 18, wherein the thickness of the supporting portion of the flexible valve is between from 0.4 times that of the first wall thickness to 1.4 times that of the first wall thickness.

20. The flexible valve of a needleless connector according to claim 17, wherein the thickness of both sides of the section with minimal thickness of the bending portion is increased respectively toward the opening end and the closed end.
